(19) United States
(12) Patent Application Publication
(10) Pub. No.: US 2016/0045191 A1
(43) Pub. Date: Feb. 18, 2016

(54) SEPTAL PERFORATION PROSTHESIS

(71) Applicant: HANSA MEDICAL PRODUCTS, INC., Carmel, IN (US)

(72) Inventors: Eric D. BLOM, Carmel, IN (US); Bryan KOSTELAC, Carmel, IN (US)

(21) Appl. No.: 14/779,736

(22) PCT Filed: Mar. 27, 2013
(86) PCT No.: PCT/US2013/034060

§ 371 Date: Sep. 24, 2015

(51) Int. Cl. A61B 17/00; B29C 31/00; A61F 2/18

(52) U.S. Cl. CPC A61B 17/0057; A61F 2/186; B29C 31/008; A61B 2017/00606; A61B 2017/00641; A61B 2017/00876; A61F 2230/0008; A61F 2230/0006; B29L 2031/753

(57) ABSTRACT

The vast majority of septal perforations are plugged by providing a septal perforation prosthesis in a limited number of flange sizes and shapes, stem sizes and stem lengths. Insertion of the prosthesis is simple and positive, and does not require, for example, folding or wadding up one flange and pulling the thus-folded or -wadded up flange through the perforation. Likewise, removal of the prosthesis does not require the occasionally traumatic pulling of a flange through the perforation.

### FIELD

This invention relates to prostheses for repairing septal perforations.

### BACKGROUND

Various types of prostheses for the repair of septal perforations are known. There are, for example, the prostheses illustrated and described in: U.S. Pat. No. 4,031,569; U.S. Pat. No. 5,968,075; DE 10 2005 021 239 A1; Anthony Products, Inc., 2008 product literature; and, Silicone Plastics, Inc. SilMedυ Nasal Septal Button 2004 product literature.

Devices for sealing a fistula in a patient's tissue are also illustrated and described in: Blom, Eric D., “Gestione clinica delle complicanze da intervento di ripristino vocale dopo fistola tracheo-esofagea,” XXII Convegno Nazionale Di Aggiornamento A. O. O. I. Impianti Fonatori Nel Laringectomizzato, Rome, Sep. 23, 1998, pp. 127-141; and, Hilgers, Frans J. M., Jessica Soolsma, Annemieke H. Ackerstaff, Fons J. M. Balm, I. Bing Tan and Michiel W. M. van den Brekel, “A Thin Tracheal Silicone Washer to Solve Periprosthetic Leakage in Laryngectomies: Direct Results and Long-Term Clinical Effects,” The Laryngoscope, vol. 118, pp. 640-645, April 2008. The disclosures of these references are hereby incorporated herein by reference. This listing is not intended as a representation that a complete search of all relevant prior art has been conducted, or that no better references than those listed exist. Nor should any such representation be inferred.

Applicant is also aware of prostheses for repairing septal perforations available from Anthony Products, Inc., 7740 Records St, Indianapolis, Ind. 46226, having flange diameters of 2.5 cm, 3 cm and 5 cm, shaft diameters of 0.3 mm-0.8 mm in 0.1 mm increments, and shaft length of 1 mm. Applicant is also aware of prostheses for repairing septal perforations available from Zomed Medical Supplies, 18370 Honeysuckle Drive, Elkhorn, NE 68022-3846, having a flange diameter of 3 cm, a shaft diameter of 5 mm and a shaft length of 3 mm. Applicant is also aware of prostheses for repairing septal perforations available from Hood Laboratories, 575 Washington Street, Pembroke, Mass. 02359, having flange diameters of 2 cm-5 cm in 1 cm increments, shaft diameters of 4, 6, 12 and 17 mm, and shaft length of 3 mm. Applicant is also aware of prostheses for repairing septal perforations available from Karl Storz GmbH & Co. KG, Mittelstrasse 8, Tuttlingen, Germany 78532, having a flange diameter of 3 cm. Typically, these devices are made of relatively low durometer silicones, with the intention being that their flanges will be trimmed to size by the clinician during fitting prior to insertion into the septal perforation. Some have even suggested trimming the outside dimensions of the stems to approximate the dimensions of the perforation to be plugged by the prosthesis. Of course, the very short lengths of the stems between the flanges of these septal perforation prostheses, for example, in the 1 mm-3mm range, make trimming of the stems difficult.

There are also the devices and methods illustrated and described in U.S. Pat. Nos. 4,402,314; 4,911,716; 5,919,231; 6,776,797; and, 7,987,851.

The disclosures of all of the above-listed references are hereby incorporated herein by reference. This listing is not intended as a representation that a complete search of all relevant prior art has been conducted, or that no better references than those listed exist. Nor should any such representation be inferred.

### SUMMARY

Applicant has determined that the vast majority of septal perforations can be plugged by providing a limited number of flange diameters, stem sizes and stem lengths. Applicant has also developed a septal perforation prosthesis which is much easier to place, and to remove, for example, for cleaning, than any other currently available prosthesis. Placement of the prosthesis of the present invention is simple and positive, and does not require, for example, folding or wadding up one flange and pulling the thus-folded or -wadded up flange through the perforation. Likewise, removal of the prosthesis, for example, for cleaning or replacement, does not require the occasionally traumatic pulling of a flange through the perforation.

A prosthesis for closing a septal perforation comprises two flanges. Each flange is provided with a raised region. Each raised region is provided with a magnet. The magnets of the two flanges are oriented so that they attract each other when the flanges are properly oriented. The raised regions cooperate in the assembled prosthesis to provide a stem which resides in the perforation. The stem has a cross-sectional configuration transverse to an axis of the stem which is one of circular and oval.

A prosthesis for closing a septal perforation comprises two flanges. Each flange is provided with a raised region. Each raised region is provided with a magnet. The magnets of the two flanges are oriented so that they attract each other when the flanges are properly oriented. The raised regions cooperate in the assembled prosthesis to provide a stem which resides in the perforation. The magnets provide an attractive force greater than about 0.2 pound force (about 0.89 N).

A prosthesis for closing a septal perforation comprises two flanges. Each flange is provided with a raised region. Each raised region is provided with a magnet. The magnets of the two flanges are oriented so that they attract each other when the flanges are properly oriented. The raised regions cooperate in the assembled prosthesis to provide a stem which resides in the perforation. The magnets are in the form of rings having facing surfaces.

A prosthesis for closing a septal perforation comprises two flanges. Each flange is provided with a raised region. Each raised region is provided with a magnet. The magnets of the two flanges are oriented so that they attract each other when the flanges are properly oriented. The raised regions cooperate in the assembled prosthesis to provide a stem which resides in the perforation. The stems have diameters greater than or equal to about 3 mm in about 1 mm increments, and less than or equal to about 19 mm.

A prosthesis for closing a septal perforation comprises two flanges. Each flange is provided with a raised region. Each raised region is provided with a magnet. The magnets of the two flanges are oriented so that they attract each other when the flanges are properly oriented. The raised regions cooperate in the assembled prosthesis to provide a stem which resides in the perforation. The stems have major×minor axis dimensions, respectively, greater than or equal to about 7 mm×about 4 mm in about 1 mm increments, and less than or equal to about 20 mm×about 15 mm.

Illustratively, the raised region is generally symmetric about the geometric center of its respective flange.

Illustratively, the spacing of the flanges in the assembled prosthesis is selected from the group consisting of about 2 mm and about 3 mm.

A method of making a prosthesis for closing a septal perforation comprises molding a low durometer silicone to form two flanges, molding a raised region on what will become a facing surface of each flange, molding a magnet into each raised region, the magnets oriented so that the two flanges, when properly oriented, attract each other.

Illustratively, molding a raised region on what will become a facing surface of each flange comprises molding a raised region having a cross-sectional configuration transverse to an axis of the raised region which is one of circular and oval.

Illustratively, the raised region is generally symmetric about the geometric center of its respective flange.

Illustratively, molding a magnet into each raised region comprises providing an attractive force greater than about 0.2 pound force (about 0.89 N) between the magnets.

Illustratively, molding a magnet into each raised region comprises molding magnets in the form of rings having facing surfaces of opposite magnetic polarity into each raised region.

Illustratively, molding a raised region on what will become a facing surface of each flange comprises molding a raised region having a cross-sectional configuration transverse to an axis of the raised region which is circular.

Illustratively, molding a raised region comprises molding a raised region having an outside diameters greater than or equal to about 3 mm in about 1 mm increments, and less than or equal to about 19 mm, and a height such that the spacing of the flanges in the assembled prosthesis is selected from the group consisting of about 2 mm and about 3 mm.

Illustratively, molding a raised region on what will become a facing surface of each flange comprises molding a raised region having a cross-sectional configuration transverse to an axis of the raised region which is oval. Illustratively, molding a raised region comprises molding a raised region having major×minor axis dimensions, respectively, greater than or equal to about 7 mm×about 4 mm in about 1 mm increments, and less than or equal to about 20 mm×about 15 mm, and a height such that the spacing of the flanges in the assembled prosthesis is selected from the group consisting of about 2 mm and about 3 mm.

### BRIEF DESCRIPTION OF THE DRAWINGS

The invention may best be understood by referring to the following detailed description and accompanying drawings which illustrate the invention. In the drawings:

FIG. 1 illustrates an embodiment of the invention;

FIG. 2 illustrates details of the embodiment illustrated in FIG. 1, taken along section lines 2-2 of FIG. 1;

FIG. 3 illustrates an embodiment of the invention;

FIG. 4 illustrates details of the embodiment illustrated in FIG. 3, taken along section lines 4-4 of FIG. 3;

FIG. 5 illustrates a partial sectional side elevational view of a nasal passageway with a device constructed according to the invention in place in the septum, taken generally along section lines 5-5 of FIG. 6;

FIG. 6 illustrates a partial sectional front elevational view of a nose with a device constructed according to the invention in place in the septum, taken generally along section lines 6-6 of FIG. 5; and,

FIG. 7 illustrates an alternative detail to the details illustrated in FIGS. 2 and 4.

### DETAILED DESCRIPTIONS OF ILLUSTRATIVE EMBODIMENTS

Clinical experience has established that, most generally, septal perforations are either round or oval in elevation. A septal perforation prosthesis having a stem in optional outside diameters greater than or equal to about 3 mm in about 1 mm increments, and less than or equal to about 19 mm would fill almost any generally round perforation encountered during this clinical experience to within a millimeter or so of its margin. A septal perforation prosthesis having optional major×minor axis dimensions, respectively, greater than or equal to about 7 mm×about 4 mm in about 1 mm increments, and less than or equal to about 20 mm×about 15 mm would fill almost any generally oval perforation encountered during this clinical experience to within a millimeter or so of its margins. In each of these sizes, flange spacings selected from the group consisting of about 2 mm and about 3 mm may be provided.

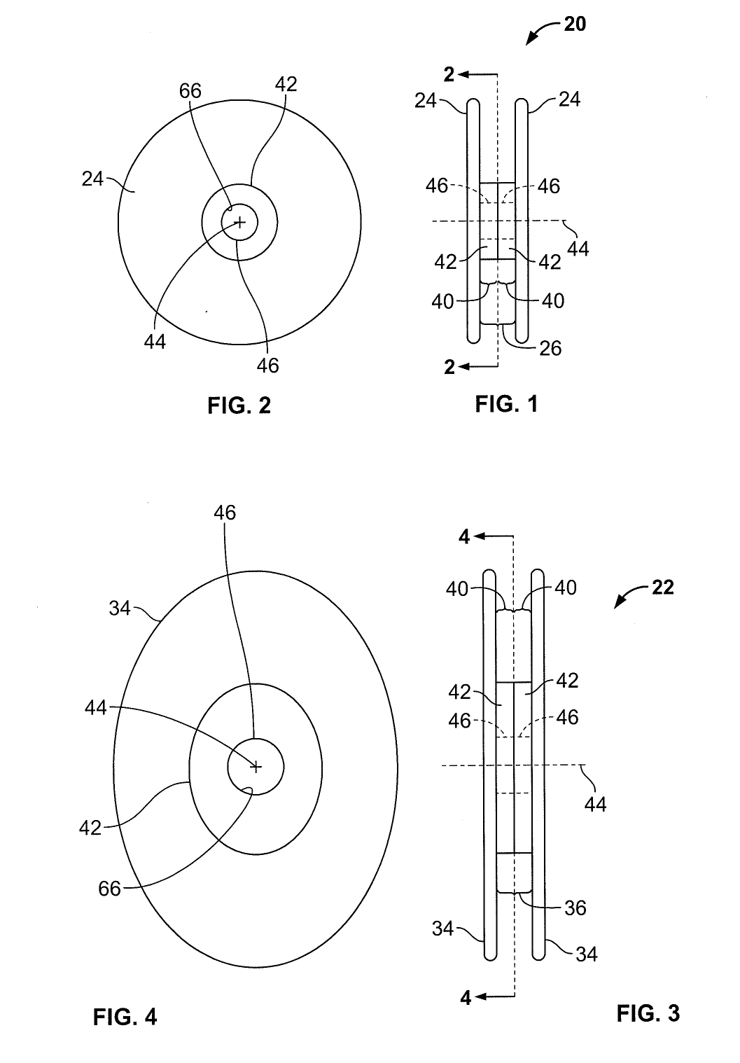

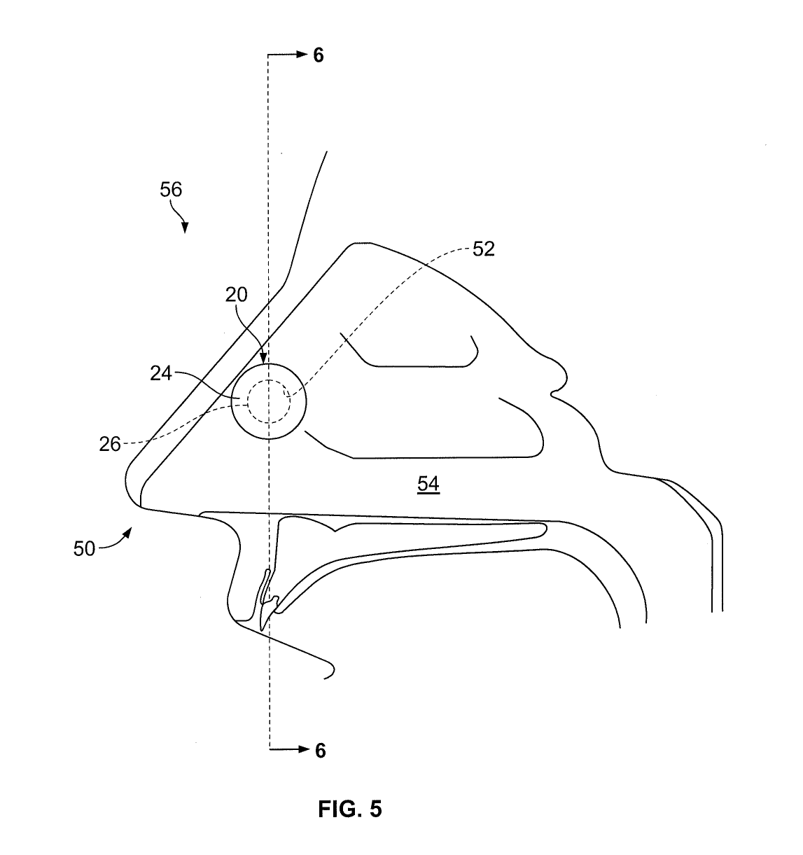

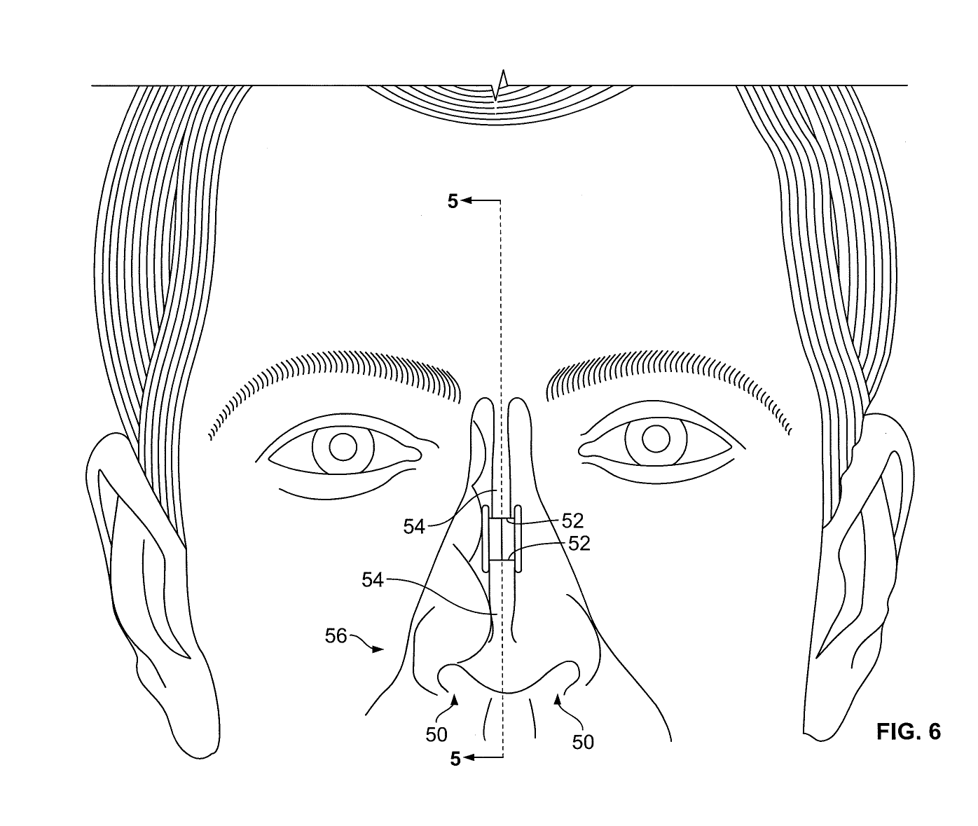

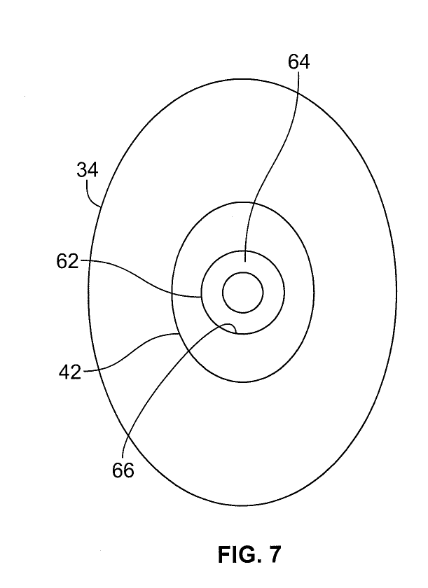

Illustratively, the prostheses 20 (FIGS. 1-2), 22 (FIGS. 3-4) come in two cross-sectional configurations, a circular one and an oval one. In the circular cross-section stem 26 configuration 20, stem 26 diameters of 13 mm, 10 mm, 7 mm and 5 mm are provided. In each of these sizes, flange 24 spacings (equivalent to stem 26 length) of 3 mm and 2 mm are provided.

In the oval cross-section stem 36 configuration 22, stem 36 major×minor axis dimensions of 21 mm×15 mm, 17 mm×13 mm, 13 mm×11 mm, 11 mm×9 mm, 9 mm×7 mm, and 7 mm×5 mm are provided. In each of these sizes, flange 34 spacings (equivalent to stem 36 length) of 3 mm and 2 mm are provided.

The stem 26, 36 portion of each prosthesis 20, 22 is divided into two lengths 40, one forming a raised region 42 symmetric about the geometric center 44 of each flange 24, 34 on one side of its respective flange 24, 34. The distance, or “overlap,” from the perimetral edge of each flange 24, 34 to the outer sidewall of the raised region 42 is about 5 mm. An illustrative flange 24, 34 thickness is in the range of about 0.030 inch (about 0.76 mm).

A magnet 46 is molded into the raised region 42. The magnets 46 of the two flanges 24 or 34 are so oriented that the flanges 24, 34 are attracted toward each other so that the flanges 24, 34, when inserted into the nostrils 50 of a wearer with the raised regions 42 facing each other, assemble themselves, owing to the magnets 46s' magnetic attraction, into the prosthesis 20, 22, with the stem 26, 36, including the coupled magnets 46, occupying the perforation 52 through the wearer's septum 54 to within a millimeter or so of the margin of the perforation 52.

In order to make the placement of the prosthesis 20, 22 suitably robust, a certain force of attraction between the magnets 46 is desired. The illustrative magnets 46 provide an attractive (and therefore a separation) force in the range of about 0.2 pound force (about 0.89 N) to about 1.0 pound force (about 4.45 N), for example, about 0.6 pound of force (about 2.67 N). Generally, force in this range protects adequately against sneezing or blowing the prosthesis 20, 22, or half of it, out of the nose 56. Force in this range also permits relatively straightforward removal of the prosthesis 20, 22 for maintenance, such as cleaning and the like, and relatively straightforward replacement once such maintenance has been performed. However, magnets providing an attractive force greater than about 0.2 pound force (about 0.89 N), and not too great as to hinder manual removal and replacement of the prosthesis for maintenance are useful. Generally this will be in the range of less than about three pounds force (about 13.35 N) or so, but may be greater.

Depending upon the magnetic material used, it may also be desirable to reduce the weight and control the strength of the magnet 46 assembly in order to keep the weight of the prosthesis 20, 22 within a range that does not distract the wearer and a strength that promotes ease of insertion and removal. In such circumstances, it may be possible to reduce the weight and strength of the magnet 46 assembly by making the magnets 46 in the form of rings 62 with their facing surfaces 64 of opposite polarity. This is illustrated in FIG. 7.

Construction of a prosthesis 20, 22 proceeds as follows. A low durometer silicone is molded to form the flanges 24, 34, each with a cup 66 (FIGS. 2, 4 and 7) provided in its raised central region 42. Silicones in the 30 durometer to 80 durometer range, for example, NuSil MED-4930 to NuSil Med-4980, are illustrative silicones for these prostheses. The magnets 46 or 62 are inserted into the cups 66, which are sized to receive the magnets 46 or 62 of the particular size and configuration for the prosthesis 20, 22 which is being constructed. Suitable magnets are neodymium/iron/boron (NdFeB) rare earth magnets available from, for example, Alliance LLC, 1450 Clark Drive, Valparaiso, Ind. 46385. Silicone, for example, the same silicone used to form the flanges 24, 34 is then placed into the cups 66 to fill whatever space remains after placement of the magnets 46 or 62 in the cups 66 and cured, providing a thin protective covering over the magnets 46 or 62. This assembly can then be coated with a thin coat of parylene or like material, if desired, to provide additional sealing away from contact with the wearer. The prosthesis 20, 22 is then ready for use.

Alternatively, the magnets 46 or 62 can be over-molded into the silicone flange 24, 34, also resulting in complete encapsulation of the magnets 46 or 62. This assembly can then be coated with parylene or like material, if desired, to provide additional sealing away from contact with the wearer.

In place of the illustrated single pole-per-pole-face magnets 46, 62, multiple pole-per-pole-face magnets can be used to aid in reducing the flux leakage from the magnet array. This may enable a smaller magnet to provide the necessary force to hold the prosthesis 20, 22 in place.

No prior art of which Applicant is aware addresses goodness of fit and ease of insertion and removal in the manner the described septal perforation prostheses do. The above noted clinical experience has established that, most generally, septal perforations are either circular or oval. While septal perforations range in size, as long as sizes of septal perforation prosthesis stems that fill a septal perforation within a millimeter or so of its margins are provided among the available septal perforation prosthesis size options, accumulation of secretions, etc., behind the flanges is not a problem. Virtually filling the perforation with the stem also results in the added benefit of a prosthesis that does not move back and forth to any noticeable extent in the perforation. The magnetic coupling/uncoupling of the flanges results in a septal perforation prosthesis that a reasonably competent wearer can insert without clinical intervention and without trauma, remove without clinical intervention or trauma, clean and replace. The prior art's need to cut, whittle, carve or otherwise reshape a flange and/or stem of a “one-size-for-all” septal perforation prosthesis is eliminated. The illustrated system thus provides: a large range of oval shafts in sizes to obturate, or fill, the perforation within 1 to 2 mm; a large range of oval flanges to completely cover the perforation; a large range of round shafts in sizes to obturate the perforation within 1 to 2 mm; a large range of round flanges to completely cover the perforation; a two part system that is easily inserted and assembled in the nose; that is magnetically coupled so it aligns itself without any additional assembly in the nose; that provides a range of flange spacings for septa of different thicknesses; that requires no trimming, cutting or fabricating by the user, that is, it is off the shelf, not custom fabricated; and, its outer aspect is constructed from biocompatible material. The illustrated system essentially eliminates prosthesis movement (perforation irritation, erosion, bleeding, crusting, discomfort, etc.) in the septum at least in the most frequently encountered sizes and shapes. It eliminates space where debris from the nose can collect. By having a comprehensive seal, obturation of the perforation and covering of the perforation, near normal nasal airflow, temperature and humidity are restored.

1. A prosthesis for closing a septal perforation comprising two flanges, each provided with a raised region, each raised region provided with a magnet, the magnets of the two flanges oriented so that they attract each other when the flanges are properly oriented, the raised regions cooperating in the assembled prosthesis to provide a stem which resides in the perforation, the stem having a cross-sectional configuration transverse to an axis of the stem which is one of circular and oval.

2. The prosthesis of claim 1 wherein the raised region is generally symmetric about the geometric center of its respective flange.

3. A prosthesis for closing a septal perforation comprising two flanges, each provided with a raised region, each raised region provided with a magnet, the magnets of the two flanges oriented so that they attract each other when the flanges are properly oriented, the raised regions cooperating in the assembled prosthesis to provide a stem which resides in the perforation, the magnets providing an attractive force greater than about 0.2 pound force (about 0.89 N).

4. The prosthesis of claim 3 wherein the raised region is generally symmetric about the geometric center of its respective flange.

5. A prosthesis for closing a septal perforation comprising two flanges, each provided with a raised region, each raised region provided with a magnet, the magnets of the two flanges oriented so that they attract each other when the flanges are properly oriented, the raised regions cooperating in the assembled prosthesis to provide a stem which resides in the perforation, the magnets being in the form of rings having facing surfaces.

6. The prosthesis of claim 5 wherein the raised region is generally symmetric about the geometric center of its respective flange.

7. A prosthesis for closing a septal perforation comprising two flanges, each provided with a raised region, each raised region provided with a magnet, the magnets of the two flanges oriented so that they attract each other when the flanges are properly oriented, the raised regions cooperating in the assembled prosthesis to provide a stem which resides in the perforation, the stems having diameters greater than or equal to about 3 mm in about 1 mm increments, and less than or equal to about 19 mm.

8. The prosthesis of claim 7 wherein the spacing of the flanges in the assembled prosthesis is selected from the group consisting of about 2 mm and about 3 MM.

9. A prosthesis for closing a septal perforation comprising two flanges, each provided with a raised region, each raised region provided with a magnet, the magnets of the two flanges oriented so that they attract each other when the flanges are properly oriented, the raised regions cooperating in the assembled prosthesis to provide a stem which resides in the perforation, the stems having major×minor axis dimensions, respectively, greater than or equal to about 7 mm×about 4 mm in about 1 mm increments, and less than or equal to about 20 mm×about 15 mm.

10. The prosthesis of claim 9 wherein the spacing of the flanges in the assembled prosthesis is selected from the group consisting of about 2 mm and about 3 mm.

11. A method of making a prosthesis for closing a septal perforation comprising molding a low durometer silicone to form two flanges, molding a raised region on what will become a facing surface of each flange, molding a magnet into each raised region, the magnets oriented so that the two flanges, when properly oriented, attract each other.

12. The method of claim 11 wherein molding a raised region on what will become a facing surface of each flange comprises molding a raised region having a cross-sectional configuration transverse to an axis of the raised region which is one of circular and oval.

13. The method of claim 12 wherein the raised region is generally symmetric about the geometric center of its respective flange.

14. The method of claim 11 wherein the raised region is generally symmetric about the geometric center of its respective flange.

15. The method of claim 11 wherein molding a magnet into each raised region comprises providing an attractive force greater than about 0.2 pound force (about 0.89 N) between the magnets.

16. The method of claim 11 wherein molding a magnet into each raised region comprises molding magnets in the form of rings having facing surfaces of opposite magnetic polarity into each raised region.

17. The method of claim 11 wherein molding a raised region on what will become a facing surface of each flange comprises molding a raised region having a cross-sectional configuration transverse to an axis of the raised region which is circular.

18. The method of claim 11 wherein molding a raised region comprises molding a raised region having an outside diameters greater than or equal to about 3 mm in about 1 mm increments, and less than or equal to about 19 mm, and a height such that the spacing of the flanges in the assembled prosthesis is selected from the group consisting of about 2 mm and about 3 mm.

19. The method of claim 11 wherein molding a raised region on what will become a facing surface of each flange comprises molding a raised region having a cross-sectional configuration transverse to an axis of the raised region which is oval.

20. The method of claim 11 wherein molding a raised region comprises molding a raised region having major×minor axis dimensions, respectively, greater than or equal to about 7 mm×about 4 mm in about 1 mm increments, and less than or equal to about 20 mm×about 15 mm, and a height such that the spacing of the flanges in the assembled prosthesis is selected from the group consisting of about 2 mm and about 3 mm.
